(19) United States
(12) Patent Application Publication
(10) Pub. No.: US 2008/0312508 A1
(43) Pub. Date: Dec. 18, 2008

(54) Vaginal Speculum Assembly

(76) Inventor: Adrian Shulman, Herzliya (IL)

Correspondence Address: YORAM TSIVION, P.O. BOX 1307, PARDES HANNA, 37111, IL

(21) Appl. No.: 10/596,439

(22) PCT Filed: Aug. 16, 2004
(86) PCT No.: PCT/IL04/00751

§ 371 Date: Jun. 14, 2006

Related U.S. Application Data
(60) Provisional application No. 60/529,100, filed on Dec. 15, 2003.

(51) Int. Cl. A61B 1/32

(52) U.S. Cl. 600/220

(57) ABSTRACT

A vaginal speculum assembly (100) including first and second speculum jaws (101, 103) to engage a vaginal aperture (402) and to provide a desired opening of the vaginal aperture (402), wherein at least one of the first and second jaws (101, 103) is associated with a mounting configuration (110) to support an inspection device (112) at a desired position relative to a vaginal cavity (404) associated with the vaginal aperture (402).

### FIELD OF THE INVENTION

The invention relates to vaginal speculums and to intra-uterine procedures.

### BACKGROUND OF THE INVENTION

Intra-uterine procedures may include Assisted Reproductive Technologies (ART) procedures, e.g., an embryo transfer during In-Vitro Fertilization (IVF) procedure or an Intra Uterine Insemination (IUI) procedure. Other procedures may include insertion or removal of an Intra-Uterine contraceptive Device (IUD), removal of an endometrial polyp, endometrial tissue biopsy, hydrography and tubal visualization, and Chronic Villi Sampling (CVS) procedures.

The intra-uterine procedures may utilize a vaginal speculum to increase an opening of a vagina to be inspected or treated. Conventional vaginal speculums may include a top blade connected, e.g., by an “O” shaped or a “U” shaped portion, to a first handle, and a lower blade connected to a second handle. The top blade may be pivotally connected to the lower blade such as to allow movement of the top blade between open and closed positions relative to the lower blade, e.g., by moving the handles together or apart.

The speculum may be inserted into a vaginal aperture when the blades are in the closed position. The blades may then be opened apart to open the vaginal aperture and allow inspecting and/or treating a vaginal cavity, e.g., by inserting one or more devices, e.g., an ultrasonic device, through the “O” shaped or “U” shaped portion, and an opening formed between the top and lower blade.

Some of the intra-uterus procedures may include opening the vaginal aperture using the vaginal speculum, inserting an ultrasound transducer into the vaginal cavity, and manually holding the transducer against the vaginal inner wall. This procedure may require the user, e.g., a physician, to manually hold the transducer accurately and firmly in order to achieve optimal results. Assistance of a second person may be required for certain procedures, for example, procedures involving the use of other operations and/or devices simultaneously with the transducer operation.

Additionally, some existing intra-uterine procedures, e.g., the IUD insertion or removal, may rely on manual (“blind”) manipulation of a surgical instrument along the uterus walls, wherein the surgeon is unable to view the uterus while performing the surgery. Other intra-uterine procedures, e.g., the CVS procedure, are performed using a transabdominal approach rather than a transcervical approach, due to the complexity of positioning the vaginal ultrasound transducer, even though the transcervical approach may be otherwise more desirable.

### SUMMARY OF EMBODIMENTS OF THE INVENTION

Embodiments of the invention may include a vaginal speculum assembly configured to enable supporting an inspection device, e.g., an ultrasound transducer, at a desired position relative to a vaginal tissue to be inspected.

According to embodiments of the invention the vaginal speculum assembly may include first and second speculum jaws to engage a vaginal aperture and to provide a desired opening of the vaginal aperture, wherein at least one of the first and second jaws is associated with a mounting configuration to support an inspection device at a desired position relative to a vaginal cavity associated with the vaginal aperture.

According to some exemplary embodiments of the invention, the first speculum jaw may include a first engagement portion and a first handle portion, and the second speculum jaw may include a second engagement portion and a second handle portion. The second jaw may be pivotally connected to the first jaw such that the first engagement portion is moveable from an open position to a closed position in relation to the second engagement portion when the first and second handle portions are moved apart.

According to one exemplary embodiment of the invention, the mounting configuration may include a mounting mechanism to support the inspection device on the first jaw. In some embodiments, the mounting mechanism may be detachable from the first jaw. For example, the first jaw may include a mounting groove able to receive a mounting portion of the mounting mechanism. The mounting mechanism may be configured to enable movement of the inspection device relative to the first jaw along at least one predetermined axis. The mounting mechanism may include a locking mechanism that, when locked, is able to prevent movement of the inspection device relative to the first jaw.

According to another exemplary embodiment of the invention, the mounting configuration may include a housing adapted to pivotably support the inspection device, wherein the housing may be connected to a shaft adapted to be inserted through a channel in the first jaw. The mounting configuration may also include a rod movable within the shaft, wherein the rod, when rotated, is able to pivotally rotate the inspection device. For example, the housing may include an adaptor to associate the inspection device with pivot portions of the housing, a base portion of the adaptor having an elongated aperture, wherein a coupler extending from a distal end of the rod may be placed in the aperture, thereby to enable the inspection device to be pivotally rotated when the rod is rotated. The mounting configuration may also include a locking mechanism that, when locked, is able to prevent movement of the shaft in relation to the channel.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features and advantages thereof, may best be understood by reference to the following detailed description when read with the accompanied drawings in which:

FIG. 1 is a schematic illustration of a vaginal speculum assembly according to one exemplary embodiment of the invention;

FIG. 2a is a schematic illustration of a mounting mechanism of the assembly of FIG. 1 in an “open” state;

FIG. 2b is a schematic illustration of the mechanism of FIG. 2a in a “closed” state supporting an inspection device of the assembly of FIG. 1;

FIG. 3 is a schematic illustration of first and second speculum jaws of the assembly of FIG. 1;

FIG. 4a is a schematic illustration of the assembly of FIG. 1 inserted into a vagina to be inspected, according to an exemplary embodiment of the invention;

FIG. 4b is a schematic illustration of the inserted assembly of FIG. 4a in an open position;

FIG. 4c is a schematic rear-view illustration of the inserted assembly of FIG. 4a;

FIG. 5 is a schematic illustration of a vaginal speculum assembly according to another embodiment of the invention;

FIG. 6 is an exploded, isometric-view of a mounting configuration according to exemplary embodiments of the invention;

FIGS. 7a and 7b are schematic, cross-section illustrations of the configuration of FIG. 6, according to exemplary embodiments of the invention; and

FIGS. 8a, 8b and 8c are schematic, side-view illustrations of the assembly of FIG. 5 in three, respective, operational positions according to exemplary embodiments of the invention.

It will be appreciated that for simplicity and clarity of illustration, elements shown in the drawings have not necessarily been drawn accurately or to scale. For example, the dimensions of some of the elements may be exaggerated relative to other elements for clarity or several physical components included in one functional block or element. Further, where considered appropriate, reference numerals may be repeated among the drawings to indicate corresponding or analogous elements. Moreover, some of the blocks depicted in the drawings may be combined into a single function.

### DETAILED DESCRIPTION OF EMBODIMENTS OF THE INVENTION

In the following detailed description, numerous specific details are set forth in order to provide a thorough understanding of the invention. However, it will be understood by those of ordinary skill in the art that the present invention may be practiced without these specific details. In other instances, well-known methods, procedures, components and structures may not have been described in detail so as not to obscure the present invention.

Embodiments of the invention may include a vaginal speculum assembly configured to enable mounting an inspection device, e.g., an ultrasound transducer, to a speculum jaw, as described below.

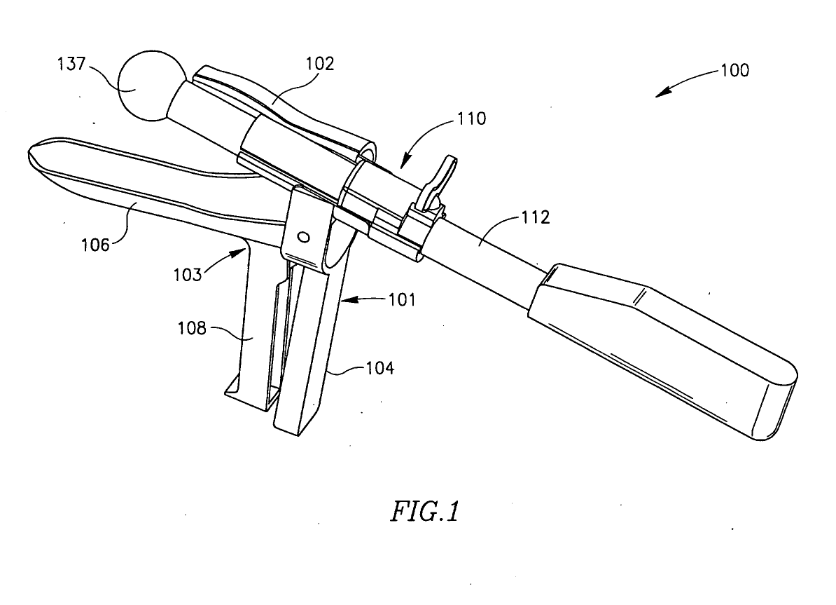

Reference is made to FIG. 1, which schematically illustrates a vaginal speculum assembly 100 according to an exemplary embodiment of the invention.

According to an exemplary embodiment of the invention, assembly 100 may include a first speculum jaw 101 having a first engagement portion 102 and a first handle portion 104, e.g., perpendicular to portion 102.

Assembly 100 may also include a second speculum jaw 103 pivotally connected to jaw 101, e.g., as described below, and having a second engagement portion 106 and a second handle portion 108, e.g., perpendicular to portion 106. According to some exemplary embodiments, portions 102 and/or 106 may be integrally connected to portions 104 and/or 108, respectively. According to other embodiments, portions 102 and 104 may include two separate elements, and/or portions 106 and 108 may include two separate elements.

According to exemplary embodiments of the invention, assembly 100 may also include a mounting mechanism 110 for mounting an inspection device, e.g., an ultrasound transducer device 112 having an ultrasound transducer head 137, to one of the speculum jaws, e.g., to jaw 101, as described in detail below.

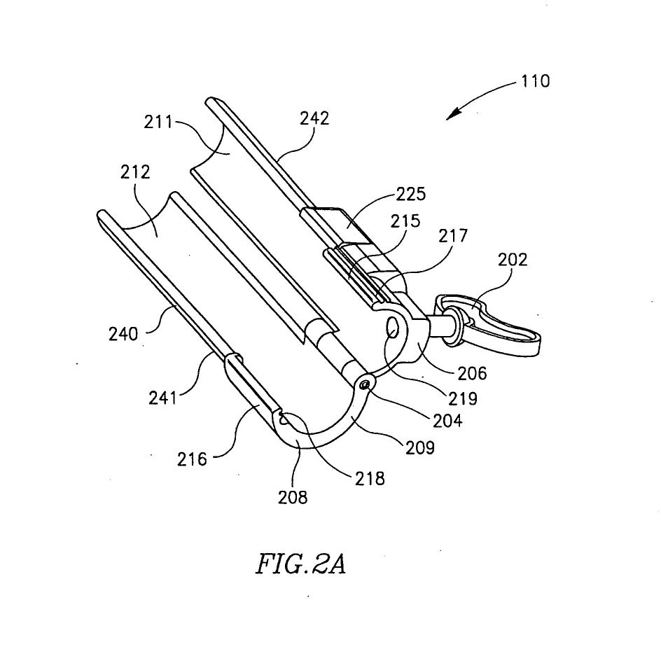

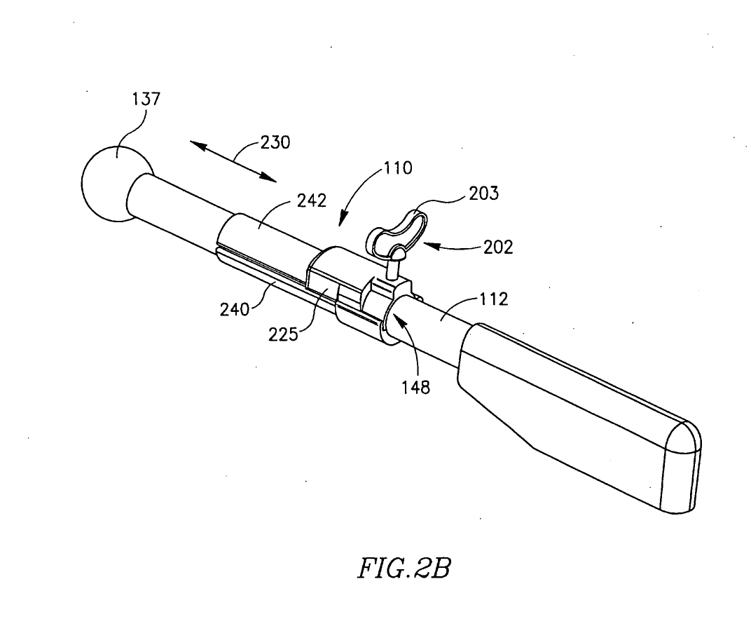

Reference is made to FIG. 2a, which schematically illustrates mechanism 110 in an “open” state, and to FIG. 2b, which schematically illustrates mechanism 110 in a “closed” state supporting transducer 112.

According to exemplary embodiments of the invention, mechanism 110 may include a first clamping element 206 pivotally connected to a second clamping element 208. For example, element 206 may be mounted on a bearing pin 204 at a first end 209 of element 208 such that clamping element 206 may be moved between open and closed positions in relation to clamping element 208.

According to exemplary embodiments of the invention, a portion 148 of transducer 112 (FIG. 1) may be placed between elements 206 and 208, e.g., in contact with an inner surface 212 of element 208, e.g., when mechanism 110 is open as shown in FIG. 2a. Element 206 may then be closed over transducer 112, as shown in FIG. 2b, e.g., to clamp portion 148 between elements 206 and 208.

According to exemplary embodiments of the invention, mechanism 110 may also include a securing configuration to secure mechanism 110 in the closed position of FIG. 2b. For example, as shown in FIG. 2a, element 206 may include a protruding portion 215 having an outer jagged surface 217, and element 208 may include a protruding portion 216 having an inner jagged surface 218. Portion 215 may be located opposite portion 216 such that jagged surface 217 may be adapted to mate with jagged surface 208, e.g., when element 206 is moved to the closed position of FIG. 2b.

According to exemplary embodiments of the invention, mechanism 110 may be configured to enable movement of transducer 112 relative to mechanism 110 along at least one predetermined axis. For example, a curvature of inner surface 211 and/or an inner surface 212 of element 208 may generally match a contour of portion 148. For example, surface 211 and/or surface 212 may have a curvature, e.g., a generally concave curvature, matching a generally round contour of portion 148. Thus, transducer 112 may be rotated, and/or moved in an “in-out” direction, e.g., as indicated by double-headed arrow 230, relative to mechanism 110

According to exemplary embodiments of the invention, mechanism 110 may include a locking mechanism, e.g., as described below, that, when locked, is able to prevent movement of transducer 112 relative to mechanism 110.

According to exemplary embodiments of the invention, the locking mechanism may include a fastener 202 fitted in a threaded aperture 219 in element 206. Fastener 202 may be fastened or released, for example, by turning a lever 203. Fastener 202 may be fastened, for example, against a segment of portion 148, such that portion 148 is forced against surface 212, thus prohibiting rotational movement and/or movement in an “in-out” direction of transducer 112 related to mechanism 110. Rotational and/or “in-out” movement of transducer 112 in relation to mechanism 110 may be allowed, e.g., when fastener 202 is released. Fastener 202 may be formed of any suitable material, for example, any suitable relatively rigid plastic material as is known in the art.

According to exemplary embodiments of the invention, mechanism 110 may also include a gripping tab 225, which may be integrally connected to element 208. Tab 225 may be used for conveniently gripping element 206, e.g., when opening and/or closing mechanism 110.

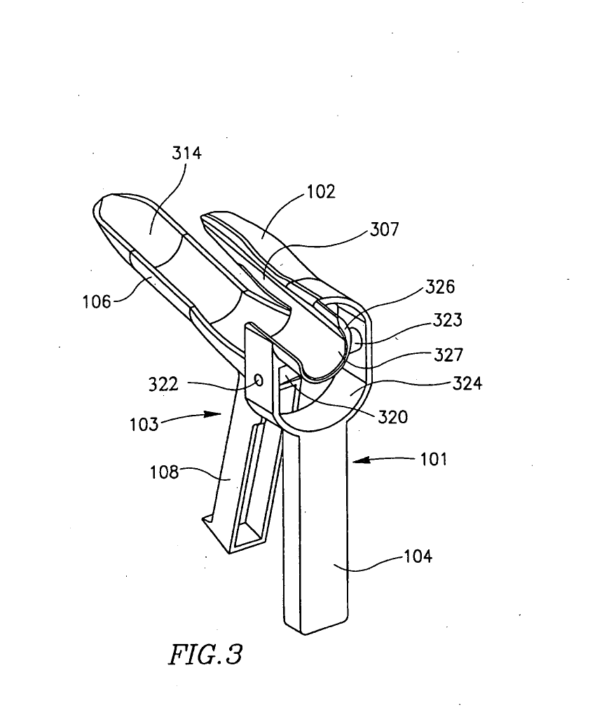

Reference is also made to FIG. 3, which schematically illustrates jaws 101 and 103 according to exemplary embodiments of the invention.

According to exemplary embodiments of the invention, jaw 103 may be pivotally connected to jaw 102, such that portion 102 may be moveable from an open position, e.g., as illustrated in FIG. 1, to a closed position, e.g., as illustrated in FIG. 3, in relation to portion 106, e.g., when portions 104 and 108 are moved apart. A pivot section 320 of jaw 103 may be pivotally connected between a first pivot section 322 and a second pivot section 323 of jaw 101. For example, section 320 may include first and second protruding ends fitted in first and second apertures or grooves of sections 322 and 323, respectively. Alternatively, section 320 may include a channel (not shown) adapted to receive a bearing pin (not shown), which may be pivotally connected to sections 322 and 323.

According to some exemplary embodiments of the invention, portion 106 may have an inner curved, e.g., concave, surface 314, e.g., as is known in the art. Jaw 101 may also include an opening 324, e.g., between portions 102 and 104, to enable a user to insert a desired device, e.g., an In-Vitro Fertilization (IVF) catheter or an Intra Uterine Insemination (IUI) catheter as are known in the art, between portions 102 and 106 when in the open position.

According to exemplary embodiments of the invention, the shape and/or dimensions of jaw 101 may be adapted for supporting mechanism 110 and/or transducer 112, as described below.

According to exemplary embodiments of the invention, jaw 101 may also include a mounting groove 326, e.g., for mounting mechanism 110. For example, groove 326 may extend outwardly in relation to portion 104, and/or may have a shape and/or size adapted to receive, e.g., and tightly support, a mounting portion of mechanism 110, e.g., when inserted therein. For example, groove 326 may have a curved, e.g., concave, surface 327 generally matching an outer surface 242 of element 206 and/or an outer surface 240 of element 208 (FIG. 2a).

According to some exemplary embodiments of the invention, portion 102 may be truncated, e.g., in relation to portion 106. For example, portion 102 may be sufficiently short so as not to obscure a “field of view” of transducer head 137, e.g., when transducer 112 is mounted on jaw 101, yet sufficiently long to engage a vagina to be inspected, as described below. Portion 102 may have an inner curved, e.g., concave, surface 307, e.g., corresponding to an outer surface of transducer 112, surface 240 and/or surface 242 (FIG. 2a).

Although some exemplary embodiments of the invention described above may refer to mechanism 110 as being detachable form jaw 101, it will be appreciated that according to other embodiments of the invention, mechanism 110 may be formed as an integral part of jaw 101.

Although exemplary embodiments of the invention are described above in the context of a mounting configuration including a mounting mechanism to mount an inspection device, e.g., device 112, to a speculum jaw, e.g., jaw 101, it will be appreciated by those skilled in the art that according to other embodiments of the invention, the inspection device may be mounted directly to the speculum jaw. For example, mounting portion 326 may have a shape corresponding to an outer surface of portion of transducer 112 such that transducer 112 may be directly mounted and secured to jaw 101.

According to exemplary embodiments of the invention, handles 104 and 108 may be interconnected, to maintain an adjustable relative position, to adjust and maintain an opening of a desired angle between portions 102 and 106. For example, handles 104 and 108 may be interconnected by a screw (not shown), as is known in the art.

According to some exemplary embodiments, the inspection device may be externally mounted to the speculum jaw, e.g., as described above. However, according to other embodiments, the speculum assembly may be modified to enable internally mounting the inspection device on the speculum jaw, e.g., to enable inserting the inspection device through opening 324 and mounting the inspection device on an inner surface of the speculum jaw.

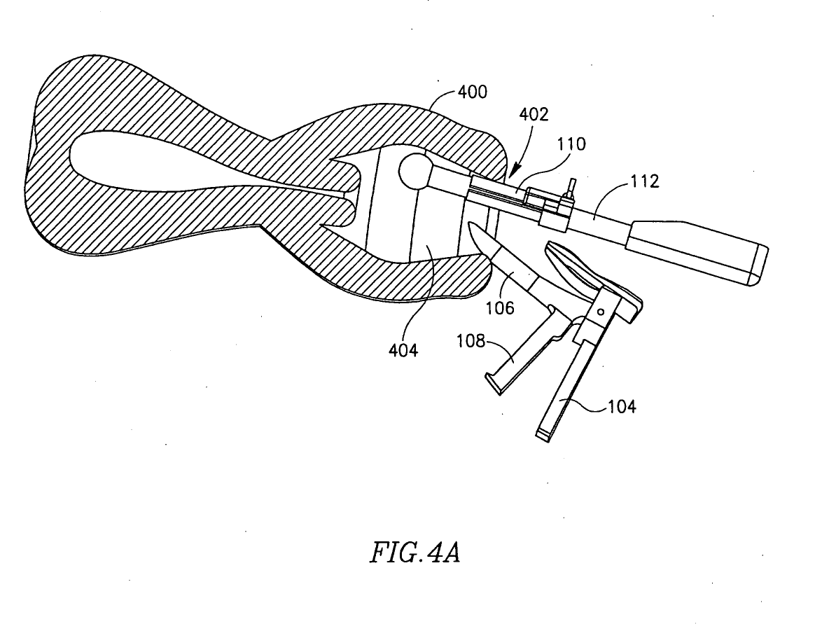

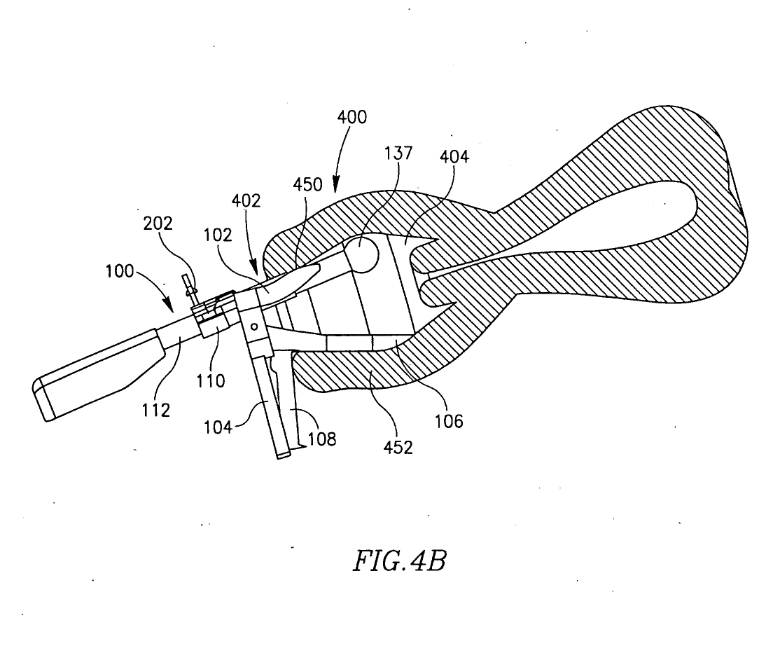

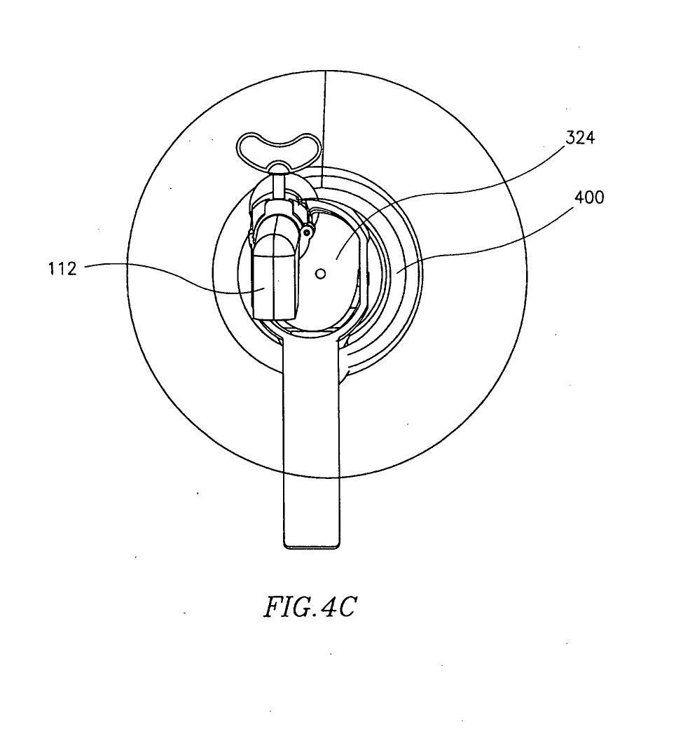

Reference is now made to FIG. 4a, which schematically illustrates assembly 100 inserted into a vagina 400 to be inspected, according to an exemplary embodiment of the invention.

According to the exemplary embodiment of FIG. 4a, transducer 112 may be clamped by mounting mechanism 110, e.g., as described above, and inserted via a vaginal aperture 402 into a vaginal cavity 404 of vagina 400. Jaws 101 and 103, e.g., when in a closed position, may then be inserted into aperture 402, and mounting mechanism 100 may be mounted on jaw 101, for example, by inserting mechanism 110 into mounting groove 326 (FIG. 3).

According to other embodiments of the invention, transducer 112 may be clamped by mechanism 110 and mounted to jaw 101 before assembly 100 is inserted into vaginal aperture 402.

After assembly 100 is inserted into aperture 402, portions 102 and 106 may be opened apart, e.g., by moving handle portions 104 and 108 towards each other.

Reference is now made to FIG. 4b, which schematically illustrates assembly 100 in an open position and engaging vagina 400 according to exemplary embodiments of the invention.

According to exemplary embodiments of the invention, engagement portion 102 may engage a first area 450 of vaginal aperture 402, and engagement portion 106 may engage a second area 452 of aperture 402. Fastener 202 may be released, e.g., in order to allow moving transducer 112 relative to vaginal cavity 404. For example, transducer 112 may be, e.g., adjustably, rotated and/or moved “in-out” relative to jaw 101. Thus, transducer head 137 may be supported at a desired position relative to vaginal cavity 404.

Reference is also made to FIG. 4c, which schematically illustrates a rear-view of assembly 100 when inserted into vagina 400.

As illustrated in FIG. 4c, opening 324 may allow insertion of any device, e.g., an IVF catheter or an IUI catheter, for engaging vagina 400, e.g., while transducer 112 is supported at the desired position relative to vagina 400.

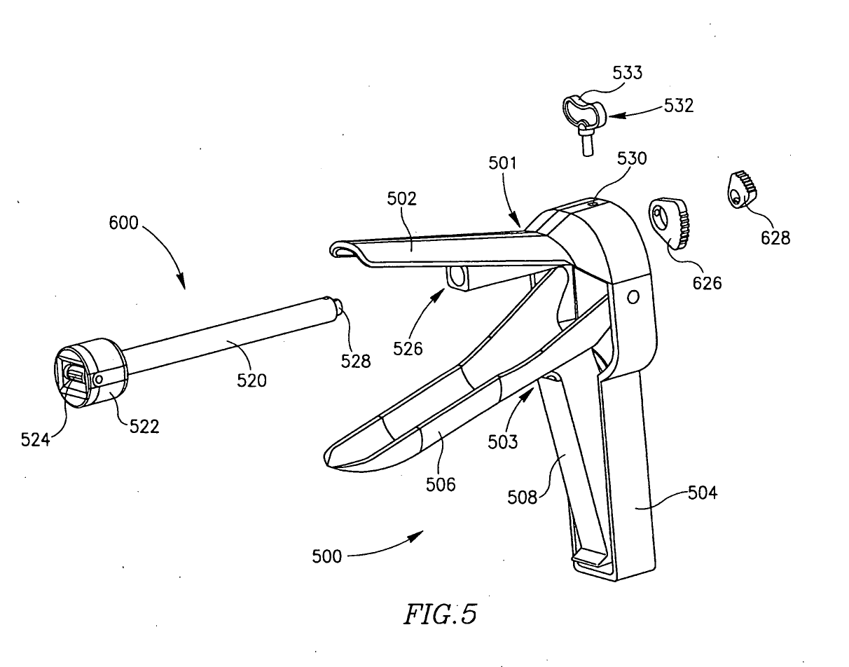

Reference is now made to FIG. 5, which schematically illustrates a vaginal speculum assembly 500 according to another embodiment of the invention.

According to the exemplary embodiment of FIG. 5, assembly 500 may include a first speculum jaw 501 having a first engagement portion 502 and a first handle portion 504, e.g., perpendicular to portion 502.

Assembly 500 may also include a second speculum jaw 503 pivotally connected to jaw 501, e.g., in analogy to the above description referring to jaws 101 and 103 (FIG. 1), and having a second engagement portion 506 and a second handle portion 508, e.g., perpendicular to portion 506.

According to exemplary embodiments of the invention, assembly 500 may also include a mounting configuration 600 for mounting an inspection device, e.g., an ultrasonic transducer 524, to jaw 501, as described in detail below.

According to exemplary embodiments of the invention, mounting configuration 600 may include a shaft 520, which may be inserted into a channel 526 associated with jaw 501. A housing 522 for an inspection element 524 may be connected to shaft 520. Inspection element 524, e.g., an ultrasonic transducer, may be pivotally mounted in housing 522 and associated with a rod 528, which may be movable within shaft 520, as described in detail below.

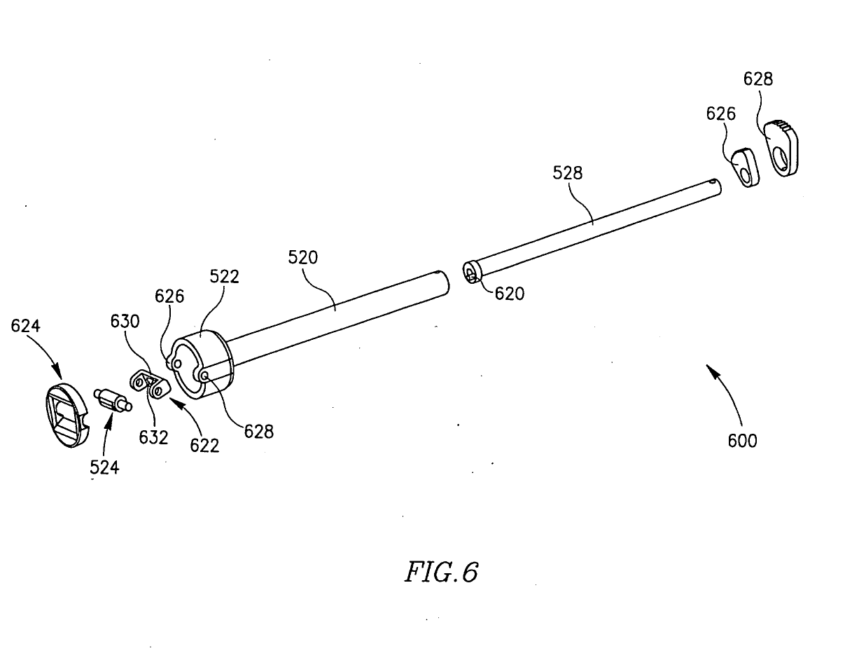

FIG. 6 is an exploded, isometric view of mounting configuration 600 according to exemplary embodiments of the invention.

According to exemplary embodiments of the invention, configuration 600 may include an adaptor 622 to connect transducer 524 to pivot portions 626 and 628 of housing 522. A base portion 630 of adaptor 622 may have an elongated aperture 632 able to receive a coupler 620 at a distal end of rod 528. Configuration 600 may also include a housing cap 624 configured to fit over housing 522, e.g., in order to protect transducer 524 and/or to secure transducer 524 to adaptor 622.

According to exemplary embodiments of the invention, transducer 524 may be pivotally rotated between “up” and “down” positions, e.g., relative to a pivot axis connecting portions 626 and 628, by rotating rod 528 relative to shaft 520, as described below.

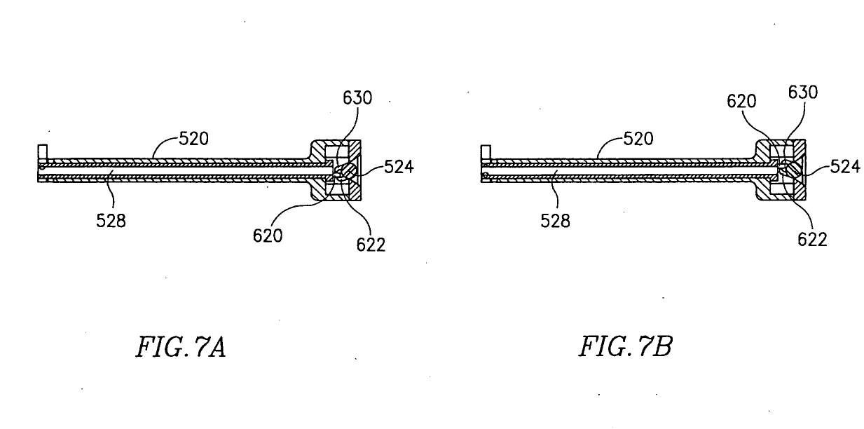

Reference is made to FIGS. 7a and 7b, which schematically illustrate a cross-section side-view of configuration 600 when transducer 524 is in “up” and “down” positions, respectively, according to exemplary embodiments of the invention.

According to exemplary embodiments of the invention, rod 528 may be rotated relative to shaft 520. Thus, coupler 620, when placed in aperture 630, may force adaptor 622 to pivotally rotate relative to the pivot axis (not shown in FIGS. 7a and 7b). As a result, transducer 524 may be forced to pivotally rotate between “up” and “down” positions, e.g., as illustrated in FIGS. 7a and 7b, respectively.

Referring back to FIG. 5, According to exemplary embodiments of the invention, transducer 524 may be mounted in housing 522, and rod 528 may be inserted into shaft 520, such that coupler 620 (FIG. 6) mates with aperture 632 (FIG. 6). Shaft 520 may then be inserted into channel 526.

According to exemplary embodiments of the invention, transducer may be, e.g., adjustably, positioned relative to jaw 501, e.g., by moving shaft 520 “in” or “out” of channel 526.

According to exemplary embodiments of the invention, assembly 600 may also include a shaft lever 626 connectable to shaft 520 and able to controllably rotate shaft 520 relative to channel 526. Thus, transducer 524, when mounted in housing 522, which is connected to shaft 520, may be controllably rotated using lever 626.

Assembly 600 may also include a rod lever 628 connectable to rod 528 and able to controllably rotate rod 528 relative to shaft 520. Thus, transducer 524 may be controllably, pivotally rotated, e.g., between “up” and “down” positions, using lever 628, e.g., as described above.

Thus, according to exemplary embodiments of the invention, transducer 524 may be controllably directed to any desired direction, e.g., using lever 626 and/or lever 628.

According to exemplary embodiments of the invention, assembly 500 may also include a locking mechanism that, when locked, is able to prevent movement of shaft 520 relative to channel 526. For example, assembly 500 may include a fastener 532 fitted in a threaded aperture 530 in jaw 501. Fastener 532 may be fastened or released, for example, by turning the fastener in the appropriate direction. Fastener 532 may be fastened, for example, against a segment of shaft 520, such that the segment is forced against channel 526, thus preventing movement of shaft 520 relative to channel 526. Rotational movement and/or “in-out” movement, i.e., movement along channel 526, of shaft 520 relative to channel 526 may be allowed, e.g., when fastener 532 is released. Fastener 532 may be formed of any suitable material, for example, a relatively rigid plastic material or metal as is known in the art.

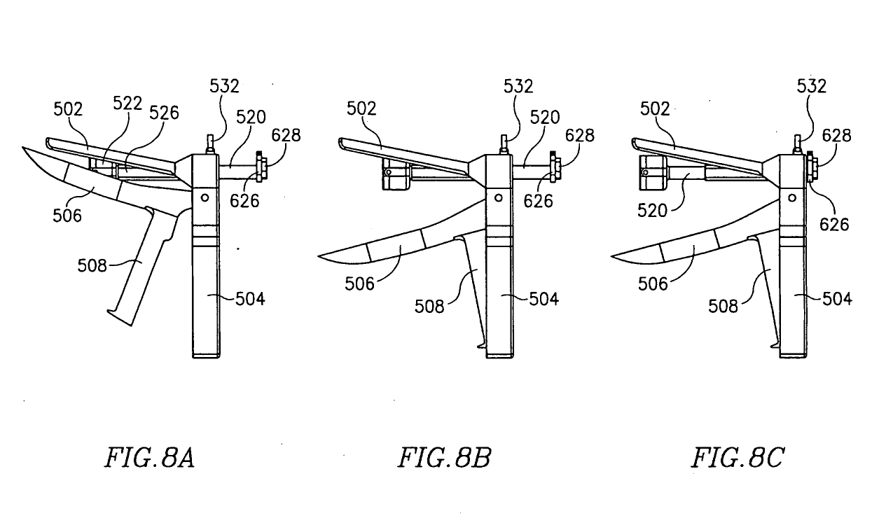

Reference is made to FIGS. 8a, 8b and 8c, which schematically illustrate a side-view of assembly 500 in three, respective, operational positions according to exemplary embodiments of the invention.

As illustrated in FIG. 8a, once shaft 520 is inserted into channel 526, shaft 520 may be “pulled-in”, e.g., such that housing 522 is relatively close to channel 526, to allow “closing” between engaging portion 502 and engaging portion 506, e.g., by separating positioning handles 504 and 508 from each other. Shaft 520 may then be locked in channel 526, e.g., by fastening fastener 532. Assembly 500 may then be inserted into a vagina to be inspected (not shown).

As illustrated by FIG. 8b, after assembly 500 is inserted into the vagina, engagement portions 502 and 506 may be separated to an open position, for example, by squeezing handles 504 and 508 towards one another.

As illustrated by FIG. 8c, fastener 532 may then be released, and shaft 520 may be pushed to extend “out” of channel 526 to a desired inspection position. Transducer 524 (not shown in FIGS. 8a-8c) may be adjustably positioned at a desired position relative to the vagina, e.g., by moving shaft 520 “in” or “out” relative to channel 526, and/or directed to a desired inspection direction, e.g. by rotating shaft 520 relative to channel 520, e.g., using lever 626, and/or rotating rod 528 relative to shaft 520, e.g., using lever 628, as described above.

It will be appreciated by those skilled in the art that a vaginal speculum assembly according to embodiments of the invention, may allow a user, e.g., a physician, to support an inspection device, e.g., an ultrasound device, at a desired position relative to a vaginal cavity. The physician may perform an intra-uterine procedure, e.g., using both hands, without being required to hold the inspection device in place during the intra-uterine procedure, as in conventional procedures. Thus, it will be appreciated by those skilled in the art that the vaginal speculum assembly according to embodiments of the invention may be conveniently used, for example, to assist in performing a Chronic Villi Sampling (CVS) procedure using a transcervical approach.

Furthermore, it will be appreciated by those skilled in the art that the speculum assembly according to embodiments of the invention may be conveniently used to assist in performing an ultrasonic guided embryo transfer.

Although, according to some embodiments of the invention described above the inspection device may be mounted onto the top speculum jaw, e.g., jaw 101 (FIG. 1) or jaw 501 (FIG. 5), it will be appreciated by those skilled in the art that, according to other embodiments, the inspection device may be mounted onto the bottom speculum jaw, e.g., jaw 103 (FIG. 1) or Jaw 503 (FIG. 5) with appropriate changes in the speculum assembly. For example, the bottom jaw may be configured to have mounting mechanisms similar to those described above in connection with the configuration of the top jaw.

Although some exemplary embodiments of the invention refer to mounting an ultrasonic transducer device to the speculum jaw, it will be appreciated by those skilled in the art the speculum assembly according to embodiments of the invention may be modified to enable mounting any other device, for example, any suitable illumination device.

While certain features of the invention have been illustrated and described herein, many modifications, substitutions, changes, and equivalents may occur to those of ordinary skill in the art. It is, therefore, to be understood that the appended claims are intended to cover all such modifications and changes as fall within the true spirit of the invention.

What is claimed is:

1. A vaginal speculum assembly comprising:
first and second speculum jaws to engage a vaginal aperture and to provide a desired opening of said vaginal aperture, wherein at least one of said first and second jaws is associated with a mounting configuration to support an inspection device at a desired position relative to a vaginal cavity associated with said vaginal aperture.

2. The speculum assembly of claim 1, wherein said first speculum jaw comprises a first engagement portion and a first handle portion, and wherein said second speculum jaw comprises a second engagement portion and a second handle portion, said second jaw being pivotally connected to said first jaw such that said first engagement portion is moveable from an open position to a closed position in relation to said second engagement portion when said first and second handle portions are moved apart.

3. The speculum assembly of claim 2, wherein said first and second handle portions are generally perpendicular to said first and second engagement portions, respectively.

4. The speculum assembly of claim 2, wherein said first engagement portion comprises a truncated engagement portion.

5. The speculum assembly of claim 1, wherein said mounting configuration comprises a mounting mechanism to support said inspection device on said first jaw.

6. The speculum assembly of claim 5, wherein said mounting mechanism is detachable from said first jaw.

7. The speculum assembly of claim 6, wherein said first jaw comprises a mounting groove able to receive amounting portion of said mounting mechanism.

8. The speculum assembly of claim 5, wherein said mounting mechanism is configured to enable movement of said inspection device relative to said first jaw along at least one predetermined axis.

9. The speculum assembly of claim 5, wherein said mounting mechanism comprises a locking mechanism that, when locked, is able to prevent movement of said inspection device relative to said first jaw.

10. The speculum assembly of claim 9, wherein said locking mechanism comprises a fastener able to secure said inspection device in said mounting mechanism.

11. The speculum assembly of claim 5, wherein said mounting mechanism comprises a clamping arrangement having a first clamping element pivotally connected to a second clamping element, wherein said clamping arrangement is able to clamp said inspection device between said first clamping element and said second clamping element.

12. The speculum assembly of claim 11, wherein said mounting mechanism comprises a securing mechanism to secure said clamping arrangement in a closed position.

13. The speculum assembly of claim 1, wherein said mounting configuration comprises:
a housing adapted to pivotably support said inspection device, wherein said housing is connected to a shaft adapted to be inserted through a channel in said first jaw.

14. The speculum assembly of claim 13 comprising a rod movable within said shaft, wherein said rod, when rotated, is able to pivotally rotate said inspection device.

15. The speculum assembly of claim 14, wherein said housing comprises an adaptor to associate said inspection device with pivot portions of said housing, a base portion of said adaptor having an elongated aperture, and wherein a coupler extending from a distal end of said rod is able to be placed in said aperture, thereby to enable said inspection device to be pivotally rotated when said rod is rotated.

16. The speculum assembly of claim 14 comprising a rod lever connectable to said rod and able to controllably rotate said rod in relation to said shaft.

17. The speculum assembly of claim 13, comprising a locking mechanism that, when locked, is able to prevent movement of said shaft in relation to said first jaw.

18. The speculum assembly of claim 17, wherein said locking mechanism comprises a fastener that, when fastened in said first jaw, is able to secure said shaft in said channel.

19. The speculum assembly of claim 13 comprising a shaft lever connectable to said shaft and able to controllably rotate said shaft in relation to said channel.

20. The speculum assembly of claim 1, wherein said inspection device comprises an ultrasound device.
